(19) United States
(12) Patent Application Publication
(10) Pub. No.: US 2025/0249158 A1
(43) Pub. Date: Aug. 7, 2025

(54) INJECTION STRETCH BLOW MOLDED PERITONEAL DIALYSIS SOLUTION CONTAINER SYSTEM

(71) Applicants: BAXTER INTERNATIONAL INC., Deerfield, IL (US); BAXTER HEALTHCARE SA, Glattpark (Opfikon) (CH)

(72) Inventors: Virgil Claudiu Manta, Overijse (BE); Mihai Alexandru Maier, Bruxelles (BE); Eric Jacques Henaut, Arquennes (BE); Davide Pozzoni, Woluwe Saint Lambert (BE); Davide Cortinovis, Bruxelles (BE); Frederic Dendal, Etterbeek (BE)

(21) Appl. No.: 18/980,922

(22) Filed: Dec. 13, 2024

Related U.S. Application Data
(60) Provisional application No. 63/550,468, filed on Feb. 6, 2024.

(51) Int. Cl. A61M 1/16; A61M 1/28

(52) U.S. Cl. CPC A61M 1/1668; A61M 1/28

(57) ABSTRACT

Peritoneal dialysis (PD) containers having an injection stretch blow molded container body, an injection site, and a pop-up outlet valve are provided. The PD containers may be included in a PD therapy system which also includes a coil conduit, patient connector and drain bag. All of these components are made from polyolefin materials. In an alternate embodiment, a two piece outlet delivery device comprising an outlet base insert and a peelable tube may be used in place of the outlet pop-up valve. Kits for the PD therapy systems are also provided.

### CROSS REFERENCE TO RELATED APPLICATIONS

The present application claims priority to U.S. Patent Application No. 63/550,468, filed on Feb. 6, 2024 the disclosure of which is incorporated herein by reference for all purposes.

### BACKGROUND

The present disclosure relates to new and improved systems and components for storing Peritoneal Dialysis (PD) solutions and the necessary accessories (Drain Bag, Coil with Patient Connector) for performing the therapy. More particularly, it relates to a system including an injection stretch blow molded (ISBM) solution container including special features for receiving new and improved inlet valves and outlet valves, and connecting them to a coil conduit with patient connector and a new and improved drain bag to provide an improved PD Therapy System.

Currently, commercial PD Therapy Systems are composed of the following components: a plastic over-pouch that protects the therapy set; a solution bag; a coil conduit with patient connector; a drain bag; and a pull-ring cap for patient connector protection. Although the commercial PD Therapy Systems are generally satisfactory, the various components are manufactured from different and distinct polymer materials which complicates logistics and production. Moreover, recycling of the disposed PD Therapy Systems sets or kits after use is difficult because of all the different parts and different materials. Alternative systems manufactured using new polymer materials that are all generally compatible for the solution container, ports, coil conduit and drain bag are needed which will simplify recycling as well.

### SUMMARY

The present disclosure provides a new and improved injection stretch blow molded PD solution container comprising: a hollow container body having a convex polyhedral configuration including a first half container body and a second opposing half container body disposed symmetrically about a central plane, each said half container body comprising a plurality of hingeably edge-connected body panels including four opposing angled rectangular side panels and four opposing angled triangular corner panels, each side panel being hingeably edge-connected to a rectangular face panel, the hollow container body having an upper end and an opposed lower end, the upper end further including an injection site; and one of the side panels adjacent the lower end of the container body further including an outlet valve-receiving shaped outlet orifice.

In accordance with a preferred embodiment, a new and improved PD solution container includes a mounted and sealed injection site with septum and a protective cap for accessing or filling the container or adding other solutions or ingredients into the container and a mounted and sealed outlet pop-up valve in the outlet valve-receiving shaped outlet orifice for emptying the container or delivering the PD solution to a patient. In accordance with this aspect, a peritoneal dialysis therapy container comprises:
a hollow container body having a convex polyhedral configuration including a first half container body and a second opposing half container body disposed symmetrically about a central plane, each said half container body comprising nine hingeably edge-connected body panels including four opposing angled side panels alternating with four opposing angled corner panels, each side panel and each corner panel being hingeably edge-connected to a face panel, directly or indirectly, the hollow container body having an upper end, an opposed lower end, and an inner fluid-receiving chamber, one of the side panels adjacent the lower end of the container body further including an outlet valve-receiving shaped outlet orifice; and the upper end further including an injection site sealingly mounted into the top of the container body; and
a pop-up outlet valve sealingly mounted in the outlet orifice, the pop-up outlet valve comprising a valve body extending axially between a container contacting end having a first diametrical dimension with an annular face and a central opening leading to an interior cavity and an opposed top wall extending parallel to and spaced from the annular face having a second smaller diametrical dimension, a double ended elongate cylindrical shaft with a central fluid passageway extending axially through the top wall from an inner end having an inner end face disposed in the cavity at a point intermediate the annular face and the top wall to an opposed outer end spaced from the top wall outside the cavity, the inner end including at least one window opening communicating with the central passageway, the valve body including a foldable collapsible peripheral sidewall bounding the cavity extending from the container contacting end to the top wall, the sidewall being movable between an inwardly folded closed position in which the inner end face is positioned adjacent the annular face in the central opening and an extended open position in which the end face is spaced inward and away from the annular face and the sidewall is outwardly unfolded, the pop-up valve further including a foil layer welded to the annular face and the inner end face in the closed position covering the central opening, the pop-up outlet valve and a foil layer being sealably welded to the outlet orifice about the annular face, whereby the pop-up valve in the closed position closes off the outlet orifice and movement of the valve body from the closed position to the open position is effective to rupture the foil between the annular face and the end face, thereby permitting fluid to flow from the container through the outlet orifice, through the ruptured foil opening into the window opening and into the central passageway to the outer end of the shaft, and into a coil conduit.

In accordance with still another aspect, the disclosure includes a kit for a peritoneal dialysis therapy system comprising a solution container containing a peritoneal dialysis therapy solution; a pop-up outlet valve attached to the solution container; a coil conduit having a first end attached to the pop-up outlet valve and an opposed end with a patient connector, the patient connector including a solution inlet conduit connected to the coil conduit and a drain conduit extending from the patient connector; and a drain bag having an internal drain reservoir connected to the drain conduit. The kit further includes a foil overpouch containing the solution container with outlet pop-up valve and injection site attached, the coil conduit with attached patient connector and the drain bag. The foil overpouch and its contents are sterilized and are maintained in sterile condition until the foil overpouch is opened.

In accordance with an alternate embodiment, instead of the pop-up outlet valve being mounted in the outlet orifice of the container, a two piece outlet device including an outlet base member insert and a peelable tube may be used as the container outlet valve. More particularly, the outlet base insert comprises an elongate cylindrical body having peripheral radial mounting flange at one end and an opposed distal end with a peripheral radial retention barb and having an inlet passageway extending therethrough. The outlet base insert is sealingly mounted in the outlet orifice. A peelable tube is provided comprising an elongate tube having first and second opposed ends, an inner fluid passageway and a crimped heat seal section disposed between the first and second ends. The peelable tube being positioned so that the first end is telescopically received over the barbed distal end of the outlet base insert member, and the crimped heat seal section and the opposed second end project outwardly from the lower end. In accordance with this outlet arrangement, the heat seal section can be uncrimped and opened to permit container solution to flow out of the orifice and into the outlet base insert and peelable tube which may in turn be connected to a coil conduit.

The system is designed in such a way that it reduces the plastic footprint of the current product, also accommodating the usage of new plastic materials for the solution bag, drain bag and the coil conduit. The system is also cheaper to manufacture, reducing the overall cost of the therapy system. The system is also scalable to include varying container sizes from 1 liter up to 6 liters and is more efficient to manufacture than available flexible bag container systems. Moreover, because all of the components are made from polyolefin materials, the system kit in accordance with this disclosure may be directly recycled without the need for special preparations.

### BRIEF DESCRIPTION OF THE DRAWINGS

FIG. 1 is an elevated plan view of a peritoneal dialysis (PD) system in accordance with the prior art.

FIG. 2 is a perspective view of the new and improved PD therapy system in accordance with the present disclosure.

FIG. 3 is a perspective view of an injection stretch blow molded (ISBM) PD solution container in accordance with this disclosure with an injection site and a pop-up outlet valve sealingly mounted to the container connected to a coil conduit.

FIG. 4 is a side elevation view of the ISBM PD solution container of FIG. 3.

FIG. 5 is exploded perspective view of the ISBM PD solution container of FIG. 3.

FIG. 6 is an elevated cross-sectional view of the PD solution container of FIG. 4, shown with a mounted and sealed outlet pop-up valve.

FIG. 7 is a perspective view of a new and improved dual chamber ISBM PD solution container in accordance with the present disclosure.

FIG. 8 is a perspective view of the pop-up valve body in accordance with the disclosure.

FIG. 9 is a perspective view of the pop-up valve body in an open condition and showing the inner end of the shaft in a retracted position in phantom.

FIG. 10 is a perspective view of the pop-up valve body of FIGS. 8-9, shown in a closed position.

FIG. 11 is a perspective view of the pop-up valve body in the closed position with a foil layer welded to the inner end of the shaft and along the annular face of the valve body.

FIG. 12 is a perspective view of the pop-up valve of FIG. 11 in the open position.

FIG. 13 is an elevated cross-sectional view of the inlet port base insert member.

FIG. 14A illustrates a peelable tube in a closed configuration, according to embodiments of the present disclosure.

FIG. 14B illustrates a peelable tube in an open configuration, according to embodiments of the present disclosure.

FIG. 15A illustrates a side profile view of a peelable tube, according to embodiments of the present disclosure.

FIG. 15B illustrates a top profile view of a peelable tube, according to embodiments of the present disclosure.

FIG. 15C illustrates section view A-A of FIG. 15A, according to embodiments of the present disclosure.

FIG. 15D illustrates section view B-B of FIG. 15A, according to embodiments of the present disclosure.

FIG. 16 is an elevated cross-sectional view of the peelable tube mounted to the outlet port base member.

FIG. 17 is a schematic view of a PD system with the ISBM container, a coil conduit with patient connector and drain conduit with a new and improved drain bag in a start of therapy condition.

FIG. 18 is a schematic view of the PD system at a later time in the therapy and showing the distention of the drain bag as it fills.

FIG. 19 is an elevated plan view of the drain bag and locking ring.

FIG. 20 is a perspective view of the locking ring.

FIGS. 21A-21C is a sequence of schematic views showing the connection of the drain bag to the drain conduit and moving the locking ring into locked position.

FIG. 22 is a schematic flow chart showing the steps of use for the new and improved PD therapy system kit in accordance with the present disclosure.

### DETAILED DESCRIPTION

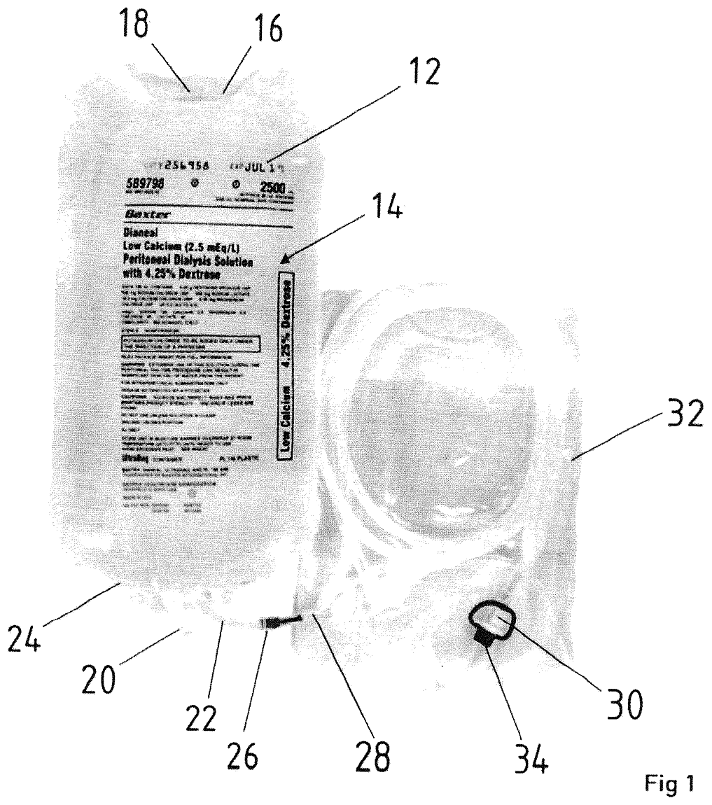

Referring now to FIG. 1, a commercial PD Therapy System kit is shown. The kit is composed of the following components: a plastic over-pouch 10 protects the components of the kit therapy set; a solution bag 12 containing a PD solution 14 is provided. Solution bag 12 has label indicia printed on a face. A hanging tab 16 with hole 18 is seen folded over at the top of the bag. Inlet port tubing 20 and outlet port tubing 22 is shown extending from a bottom edge seam 24. A green connector 26 is shown connecting the bag outlet tube 22 to a coil conduit 28 with patient connector 30 is provided with the kit which also includes a drain bag 32 and a pull-ring cap 34 for patient connector protection. Frequently, each of these separate components comprise different polymer compositions which dramatically increases the complexity of sourcing each of the different parts and materials, as well as raw materials supplying the various ingredients and managing supply chain concerns.

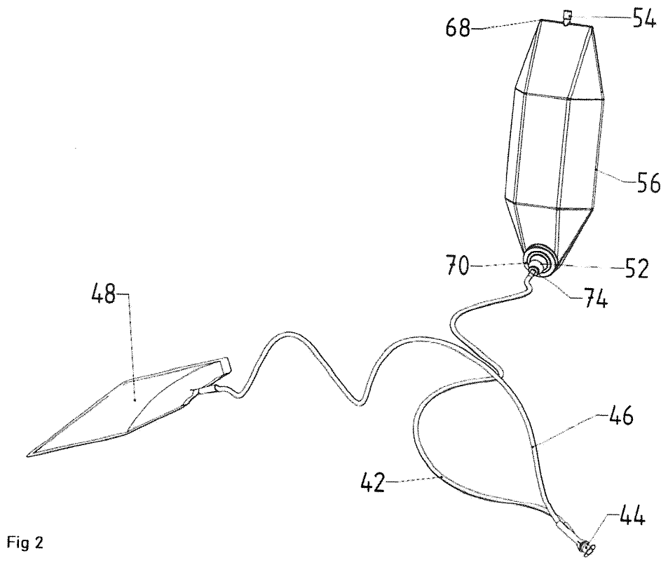

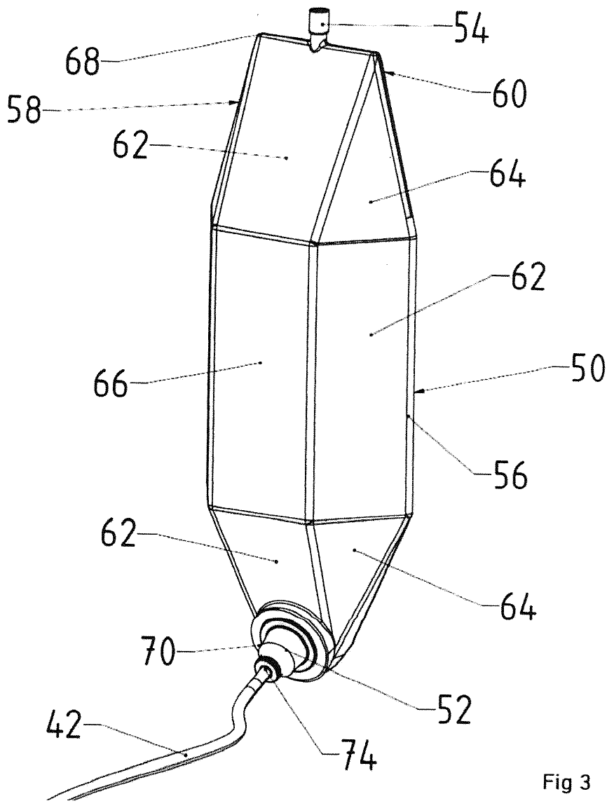

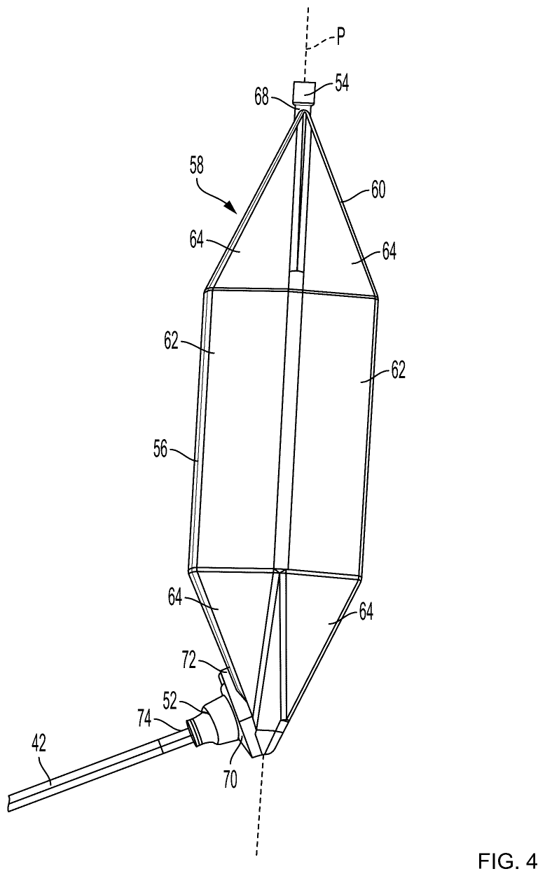

FIG. 2 shows the new and improved PD therapy system 40 and in accordance with an embodiment of the present disclosure. System 40 includes a coil conduit 42 with patient connector 44 and a drain conduit 46 connected to a drain bag 48. A new and improved injection stretch blow molded solution container 50 in accordance with an embodiment of the disclosure shown in FIGS. 2-3 includes an outlet pop-up valve 52 and an injection site 54. Solution container 50 comprises a hollow container body 56 having a convex polyhedral configuration as shown in FIGS. 3-6, including a first half container body 58 and a second opposing half container body 60 disposed symmetrically about a central plane, P. Each half container body 58, 60 comprises a plurality of hingeably edge-connected body panels including four opposing angled rectangular side panels 62 and four opposing angled triangular corner panels 64. Each side panel 62 is hingeably edge-connected to a rectangular face panel 66. The hollow container body 56 has an upper or top end 68 and an opposed lower or bottom end 70. The injection site 54 extends from the upper end 68. One of the side panels 62 adjacent the lower end 70 of the container body 56 includes a pop-up outlet valve 52 mounted to an outlet pop-up valve receiving shaped outlet orifice 72 as shown in FIG. 3. Pop-up outlet valve 52 has an outlet end 74 connected to coil conduit 42.

The ISBM container 50 is injection stretch blow molded from a pre-form in accordance with methods known to those skilled in the art from a polyolefin polymer and preferably container 50 comprises polypropylene. Preferably container body 50 comprises Bormed RD808CF™ (polypropylene).

Container 50 is designed to be self-collapsing as is known to those skilled in this art and this feature aids in emptying the container in a manner also known to those skilled in this art.

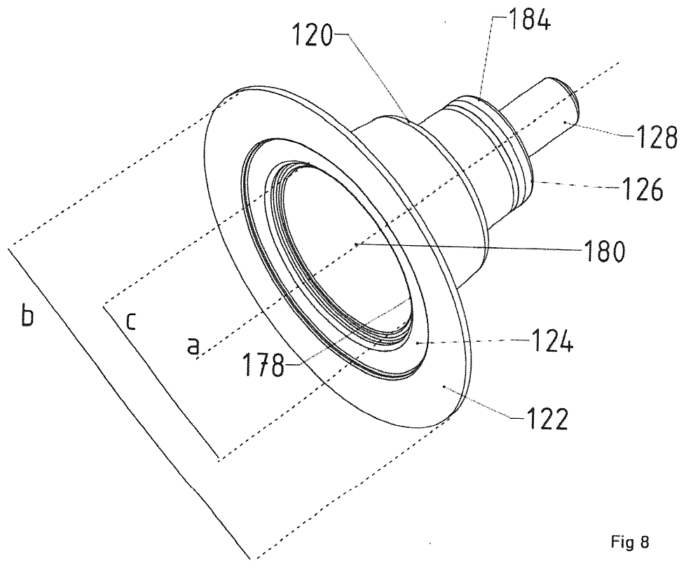

Turning now to FIGS. 8-12, the new and improved outlet pop-up valve 52 in accordance with preferred embodiment is shown. Pop-up valve 52 includes an elongate bell-shaped valve body 120 having a first container contacting end 122 including an annular face 124 with a central opening 178 leading to an inner cavity 180. A top wall 184 extends generally parallel to and spaced from the container contacting end 122. The top wall 184 has a diametrical dimension c that is smaller than the diametrical dimension b of container contacting end 22. Pop-up valve body 120 is generally symmetrical about a longitudinal axis, a. An elongate double ended shaft 182 extends axially through top wall 184 from an inner end 174 having an inner end face 188 disposed in cavity 180 at a point intermediate annular face 124 and top wall 184 to an outer end 128 which extends beyond top wall 184 located outside of cavity 180. Shaft 182 has a central passageway 176 extending therethrough for conducting fluids along the length of the shaft and to a central exit opening 178 in the outer end 128. Inner end 174 includes at least one fluid flow window opening 190 disposed between the inner end 174 and top wall 184 communicating with central passageway 176.

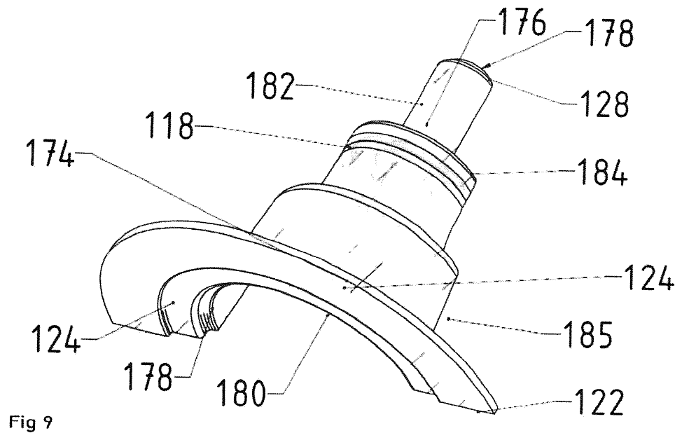

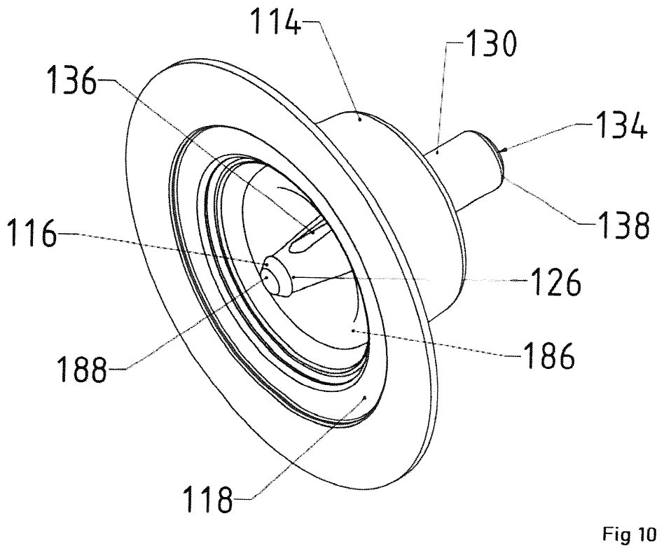

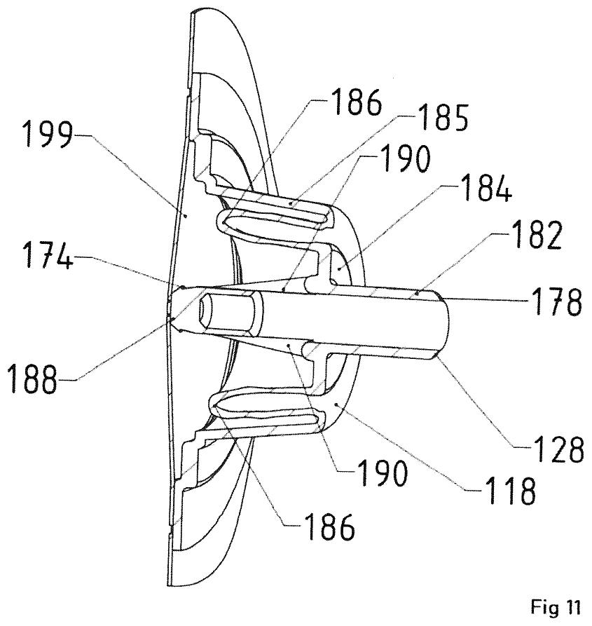

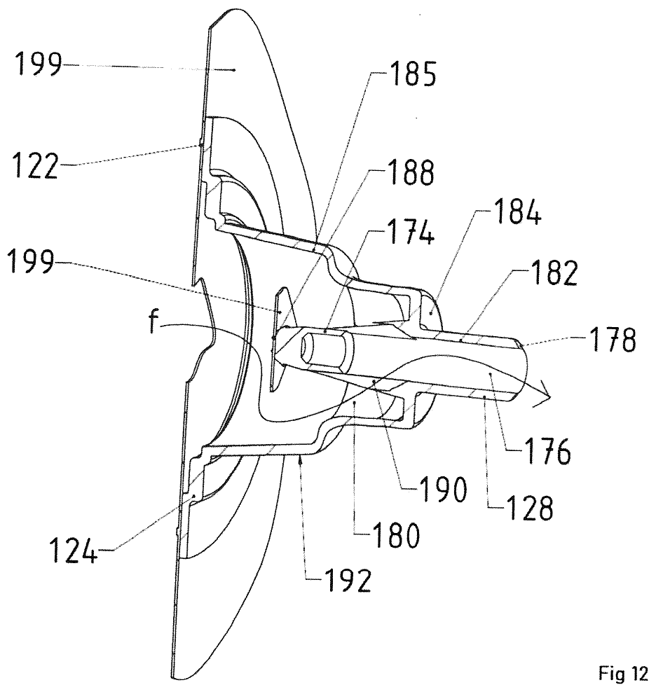

Valve body 120 includes a foldable collapsible peripheral sidewall 185 bounding cavity 180 extending between container contacting end 122 and top wall 184. Sidewall 185 is movable between an inwardly folded and closed position as shown in FIGS. 10-11. In the closed position the inner end face 188 of shaft 182 is positioned adjacent the annular face 124 in the central opening 178. In the closed position top wall 184 is advanced forwardly to lie relatively closer to container contacting end 122 and sidewall 185 is folded as shown at 186. Sidewall 185 is also movable to an extended open position as shown in FIGS. 8-9 and 12, In the open position, inner end face 188 is spaced away from annular face 124 to lie inwardly within the cavity 180 and top wall 184 is moved relatively outwardly from the container contacting end 122. Sidewall 185 is no longer collapsed and folded but is now unfolded and extended as indicated at 192 in FIG. 12.

Container pop-up valve 52 in the closed position has its central opening 178 sealed or closed by a foil layer 199 as shown in FIG. 11. Foil layer 199 is welded to valve body 120 about annular face 124 and inner end face 188. In this sealed condition, pop-up valve 52 may be sterilized, packaged and shipped for later affixation to a container.

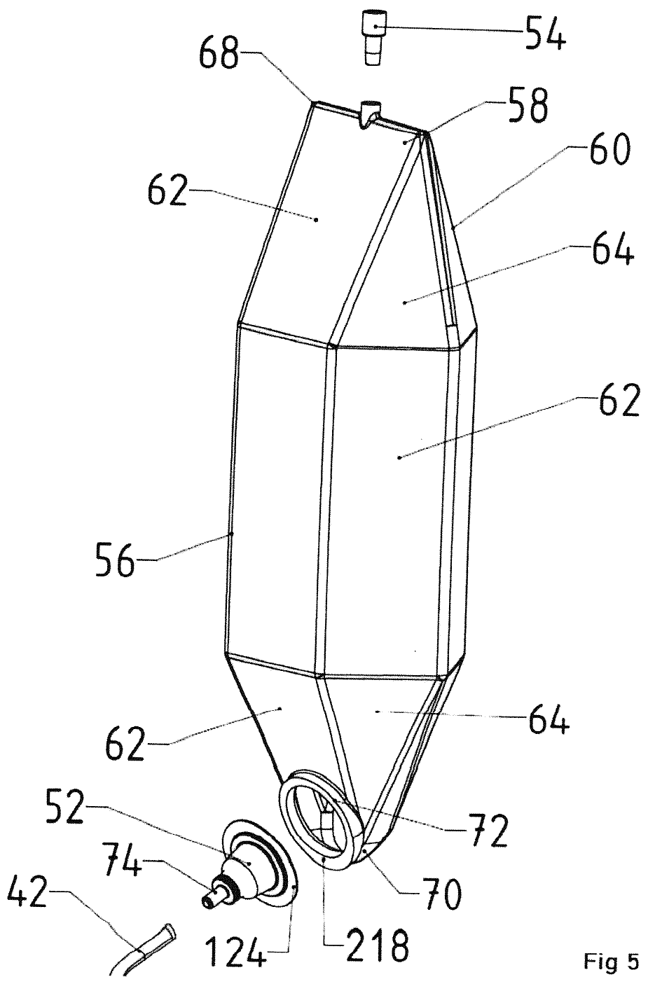

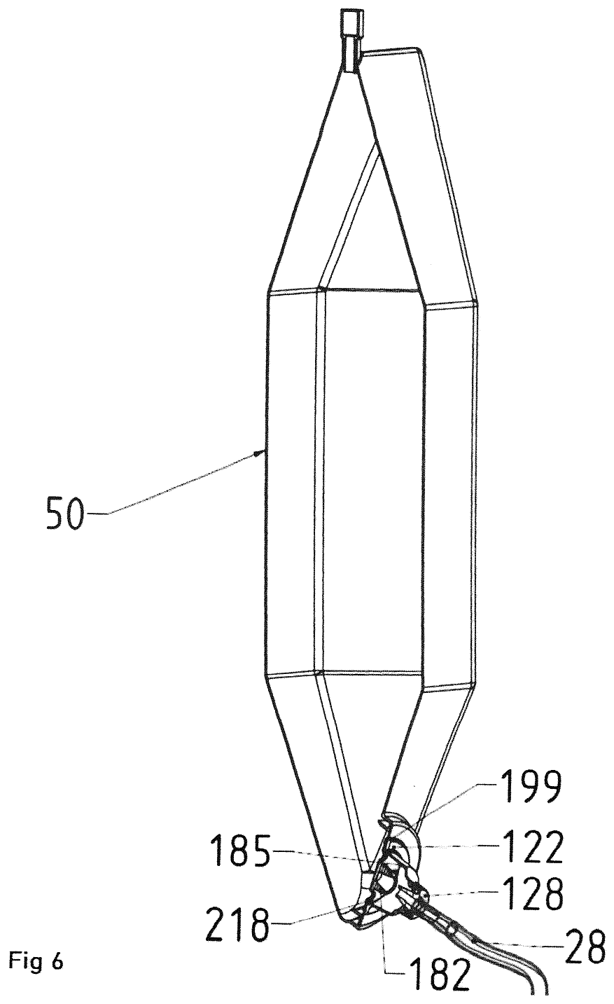

In accordance with a preferred embodiment pop-up valve 52 is welded to the outlet valve-receiving shaped outlet orifice 72 provided in the container body 56. Pop-up valve 52 is placed so that the foil layer 199 and annular face 124 are positioned in face to face abutting contact with mounting surface 125 in the shaped outlet orifice 72 as shown in FIGS. 5-6. The pop-up valve 52 is sealed to the shaped outlet orifice 72 by welding all along the annular face 124 and foil layer 199 and mounting surface 125. Heat welding is effective to sealingly bond the annular face 124, foil layer 199 and shaped outlet orifice 72 together. Pop-up valve 52 will remain in the closed position unless it is forceably activated by pulling on outer end 128 (or by pulling on a coil conduit connected to outer end 128) to flip the closure from the closed position to the open position. The act of moving the closure from the closed to the open position is effective to rupture the foil layer 199 between the annular face 124 weld and the end face 188 weld as shown in FIG. 12. In the open position shown in FIG. 12, container fluid flows through the ruptured film opening around retracted inner end 174 into the side fluid flow window openings 190 on shaft 182 into the central passageway 176, out through central opening 178 and out into a coil conduit 42 connected to outer end 128. This flow path is indicated by arrow f as shown in FIG. 12.

In accordance with the preferred first embodiment, the valve body 120 comprises a one-piece unitary integral shaped molded part. Preferably the valve material is a thermoplastic elastomer (TPE) compatible with polyolefin container films. These materials are well known to those skilled in this art.

The TPE material selected should have the required flexibility and toughness when molded into the bell shape and be compatible with the container materials as well as the medical fluids. The outlet pop-up valve 52 in accordance with the embodiment is especially adapted for use on containers comprising polypropylene. This is because the valve 52 is designed to break the foil layer film 199 in use. Foil layer 199 may comprise aluminum foil or a polypropylene film. Polypropylene containing films upon rupturing do not fragment into particles. Other container films, especially those comprising polycarbonate, fragment into particles which could contaminate the medical fluid which is unacceptable or the fragments may occlude the flow through the outlet closure which is also unacceptable. In preferred embodiments, the container sidewall film comprises polypropylene and the pop-up valve 52 comprises a blend of polypropylene and polyamide, especially preferably 75% of Bormed RD808CF™ (polypropylene) and 25% of Kraiburg TM5MHD S100B™ (polyamide). In other preferred embodiments, the container sidewall film comprises polyethylene and the outlet pop-up valve comprises a blend of polyethylene, polyamide and polyacrylonitrile. In either case, these materials are selected so that they can be recycled together which means the PD therapy system may be simply disposed of after the container is emptied.

The withdrawal force that must be exerted on the coil conduit 28 and outer end 128 of the shaft 182 should not be so low as to permit unintentional opening of the pop-up valve. The withdrawal force should not be so high that medical personnel are unable to move the pop-up valve from the closed position to the open position by pulling outwardly on the outer end of the shaft 182 or a coil conduit 42 connected to the outer end 128. The combination of the tear strength of the foil layer 199 and a preload force of the valve body 120 which must be overcome to flip the pop-up valve from closed to open and maintain it in the open position all factor into calculating the withdrawal force desired. Typical withdrawal forces for the container pop-up valve 52 are from about 3 to 10 psi (0.2 to 0.7 kg/cm2). Preferably the displacement distance to activate the pop-up valve from closed to open position is from about 5 to about 20 mm, especially preferably about 10 mm.

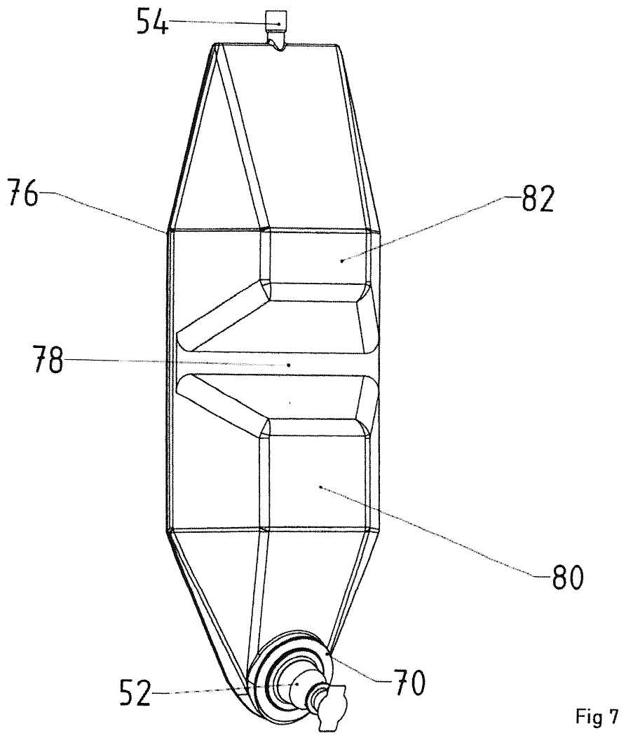

The new and improved ISBM container may also be provided as a dual chamber container 76 shown in FIG. 7. In accordance with methods well known to those skilled in this art, a peel seal 132 is provided at an intermediate point along the length of the container 56 which effectively subdivides the container 56 into a first chamber 134 and a second chamber 136. In this manner, two components may be separated until ready for therapy. The peel seal 132 is opened by applying pressure to either chamber to open the peel seal to permit mixing of the components in a well-known manner. Thereafter, pop-up valve 52 may be activated to permit the mixed components to be delivered to the coil conduit 42.

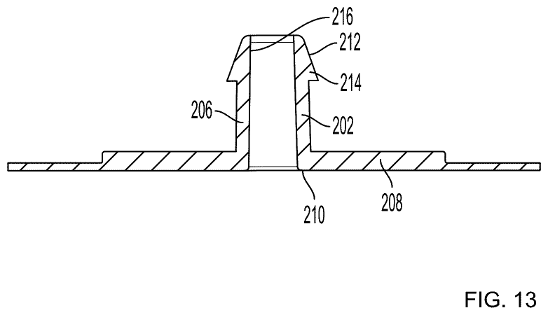

In accordance with an alternate embodiment shown in FIGS. 13-16, instead of the pop-up outlet valve 52 being mounted in the outlet orifice 72 of the container 50, a two piece outlet device 200 including an outlet base member insert 202 and a peelable tube 300 may be used as the container outlet valve. More particularly, the outlet base insert comprises an elongate cylindrical body 206 having peripheral radial mounting flange 208 at one end 210 and an opposed distal end 212 with a peripheral radial retention barb 214 and having an inlet passageway 216 extending therethrough. The outlet base insert 202 is sealingly mounted in the outlet orifice 72 by welding mounting flange 208 to the circular mounting surface 218 provided by the outlet orifice 72 to seal the outlet base member insert 202 to the container 50.

Peelable tube 300, in which a single use valve is provided is a sterile, user friendly, single use valve that reduces material cost and environmental impact compared to frangible and peelable seal systems currently used with medical solution bags. The peelable tube 300 described herein provides a user friendly advantage over previous systems, as operation simply requires a pinching action from a user to operate the valve. Production advantages are also provided by the peelable tube 300, such that production of the valve simply requires a section of compatible tubing and an apparatus with which to form the heat seal (e.g., via a heat weld).

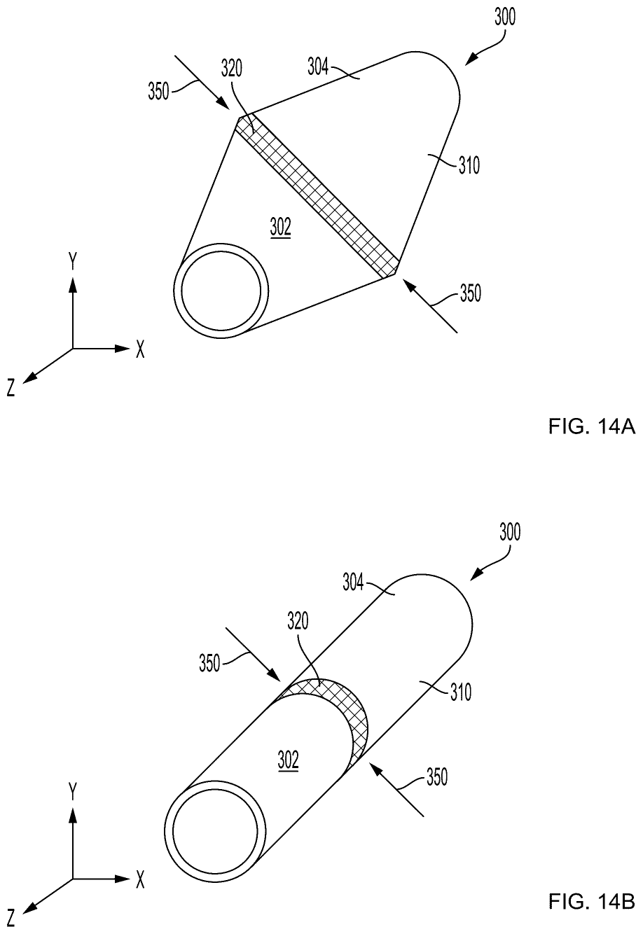

FIG. 14A shows a peelable tube 300, in the closed configuration consisting of a section of tube 310 and a heat seal 320 formed in the section of tube 310, according to embodiments of the present disclosure. The peelable tube 300 has a first end 302 and a second end 304. The heat seal 320 blocks fluid communication between the first end 302 and the second end 304. As compressive forces 350 are applied perpendicularly to the extremities of the heat seal 320, the heat seal 320 is disengaged by an inner layer “peeling” apart from itself, thereby allowing the peelable tube 300 to transition to an open configuration, (see, FIG. 14B) where fluid communication through the peelable tube 300 is facilitated. When closed and blocking fluid communication between the first end 302 and second end 304, the heat seal 320 is capable of resisting substantial internal hydraulic forces of fluids in the tube 310, while simultaneously requiring comparably little compressive force 350 to disengage the heat seal 320 and thereby permit fluid communication through the peelable tube 300.

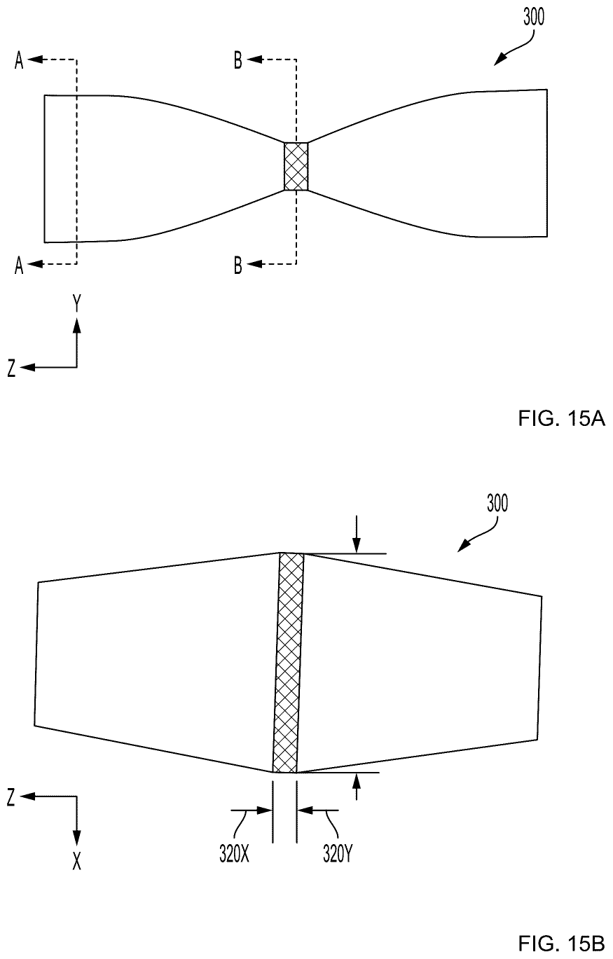

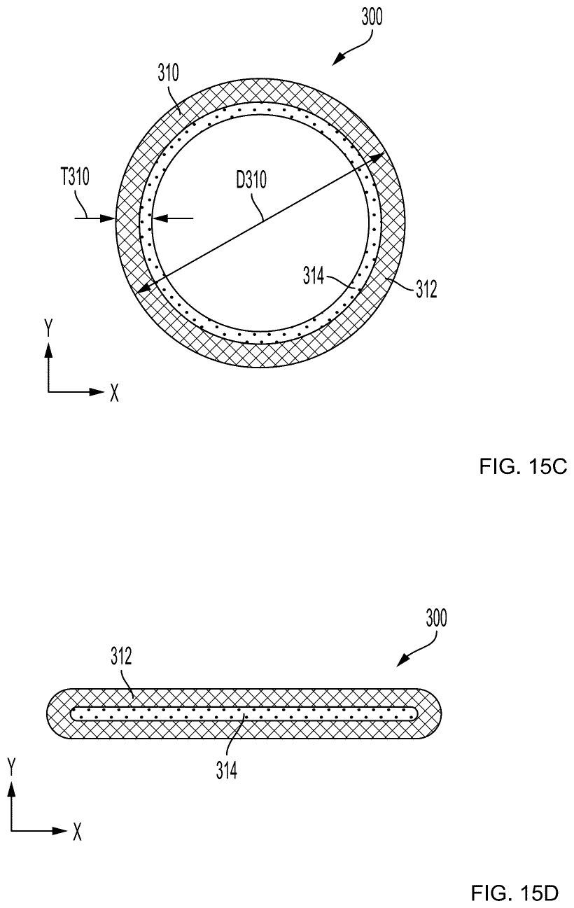

FIG. 14B illustrates a peelable tube 300 in an open configuration, according to embodiments of the present disclosure. In some examples, the heat seal 320 is disengaged in response to a pinching motion, the action from which induces the thermally bonded inner layers (see, FIG. 15C) of the tube 310 to unpeel from one another to thereby re-establish fluid communication between the first end 302 and the second end 304, previously blocked by the closed heat seal 320.

In various embodiments, the outlet device 200 may be used with various therapeutic systems, including at-home medical therapeutic systems. As such, the operators of the peelable tube 300 may include patients or their caretakers, who may not be medical professionals. Accordingly, the peelable tube 300 provides a robust and easy-to-use design. In various embodiments, the force 350 used to pinch the heat seal 320 when opening the peelable tube 300 is about 2 Newtons (N), about 4 N, about 6 N, about 8 N, about 10 N, about 15 N, about 20 N, or about 30 N.

According to some embodiments, the ratio of a magnitude of the minimum compressive pinching force 350 required to disengage the heat seal 320 relative to the fluid pressure force per square centimeter that the heat seal 320 can withstand is about 1:25, about 1:50, about 1:100, or about 1:250 (although higher forces 350 may also be applied to disengage the heat seal 320).

FIG. 15A shows a planar view of the peelable tube 300, according to embodiments of the present disclosure. In various embodiments, the heat seal 320 is formed by crimping, or applying a compressive force, to an outer circumference of the tube 310 and applying heat to fully or partially melt the material of the tube 310 and cause the inner lining (see, FIG. 15C) of the tube 310 to deform and fuse together. After the fusion, the tube 310 remains compressed and the fluid pathway through the tube is closed, preventing fluid communication through the peelable tube 300 once the inner lining re-solidifies.

In some embodiments, the heat seal 320 is selectively disengaged by a user applying a pinching action to the extremities of the heat seal 320, perpendicular to the direction in which the heat seal 320 was formed.

FIG. 15B shows a top planar view of the peelable tube 300, according to embodiments of the present disclosure. As the tube 310 is compressed in a first direction to form the heat seal 320, the tube 110 expands in a perpendicular direction, such that the width Y320 of the heat seal 320 is greater than the diameter D310 (see, FIG. 15C) of the tube 310.

According to some embodiments, the length X320 of the heat seal 320, taken along a longitudinal axis of the tube 310, is positively correlated with the amount of fluid pressure the heat seal 320 can withstand in the tube 310. In some embodiments, the heat seal 320 can withstand at least about 50 kilopascals (kPa), about 100 kPa, about 250 kPa, about 500 kPa, or about 1 megapascal (MPa).

FIG. 15C illustrates a sectional view of the peelable tube 100 of FIG. 15A along section A-A, including inner layer 314 and outer layer 312, according to embodiments of the present disclosure.

According to some embodiments, the tube 310 is composed of one or multiple thermoplastics, including an inner layer 314 which is constructed of a thermoplastic with a lower melting point than that of the thermoplastic from which the one or more outer layers 312 of the tube 310 is/are constructed. As the heat seal 320 is formed, the heat and crimping force deforms the tube 310 and melts the inner layer 314 to itself. The inner layer 314 melts and forms an adhesive that bonds the tube 310 closed, while still maintaining laminar connection to the outer layers 312. The inner layer 314 bonds to itself when a crimping force is applied to the tube 310 and the material of the inner layer 314 is allowed to cool.

According to some embodiments, the applied heat is sufficient to melt the inner layer 314, but not the outer layer 312. In some examples, the heat is applied at about 80 degrees Celsius (C), about 85 degrees C., about 90 degrees C., about 95 degrees C., or about 100 degrees C. In other examples the heat is applied at about 100 degrees C., about 110 degrees C., about 120 degrees C., about 130 degrees C., about 140 degrees C., about 150 degrees C., about 160 degrees C., or about 170 degrees C.

In various embodiments, the outer layer 312 is made of a first thermoplastic different from a second thermoplastic from which the inner layer 314 is made from. For example, the layers may include one or more or blends of: polypropylene, polyethylene, Acrylonitrile Butadiene Styrene (ABS), and various other thermoplastics and bonders, some of which may be clear or transparent, allowing an operator to observe fluid flow through the tube 300, and where (and to what extent) the heat seal 320 has been formed in the tube 300. In some examples, inner layer 314 contains a thermochromic element. When the heat seal 320 is formed, the thermochromic element reacts to the heat and changes a color state. This change in color state can be used to visually indicate that the heat seal 320 has been properly formed. The change in color state may include a visible change in hue, chroma, opacity, or combinations thereof.

FIG. 15D illustrates a sectional view of the peelable tube 300 of FIG. 15A along section B-B, according to embodiments of the present disclosure. Section B-B is taken across the heat seal 320, where the inner layer 314 has been fused together to form a bond.

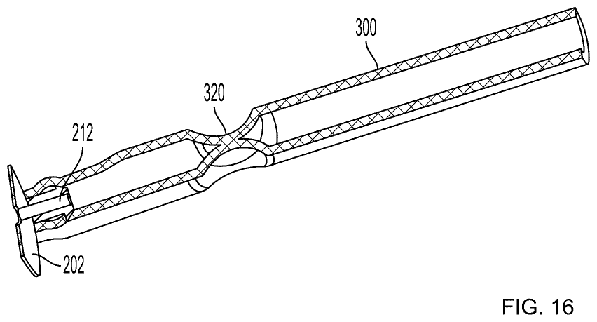

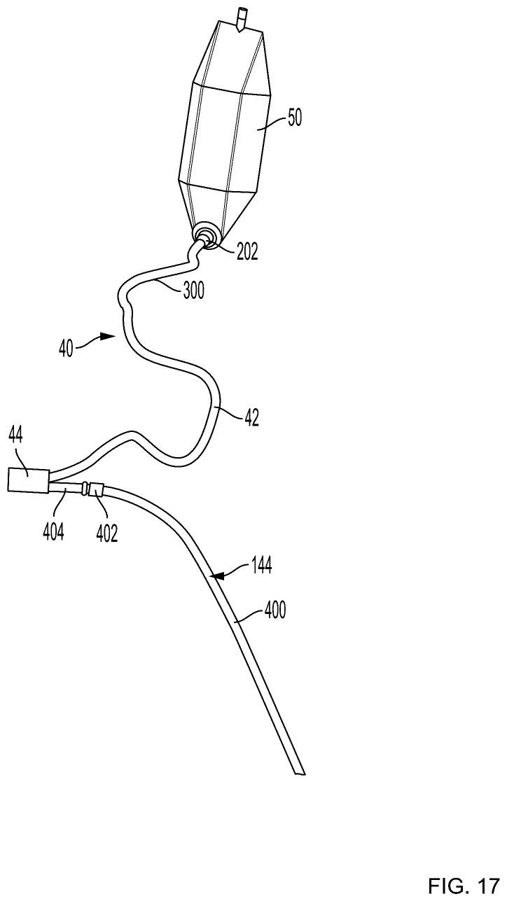

FIGS. 16 and 17 shows the peelable tube 300 installed in a fluid delivery system, including a medical solution container 50, with outlet base member 202 and tubing 310, according to embodiments of the present disclosure. The peelable tube 300 is connected on one end to the medical solution container 50, as an example fluid reservoir. More particularly, as shown in FIG. 16, peelable tube 300 is telescopically positioned on the distal end 212 of outlet base member 202 and the other end of the peelable tube 300 is connected to the tubing 310. The heat seal 320 on the peelable tube 300 has sufficient strength to resist the static hydraulic forces of the fluid contained in the medical solution container 50. When the peelable tube is opened, the fluid flows from the container 50 through the peelable tube 300 into the tubing 310. The medical solution container 50 is one example of a reservoir configured to hold a medical fluid, but the present disclosure contemplates other potential uses for the peelable tube 300 (e.g., automotive fluids, cleaning solutions, potable liquids, etc.).

In some embodiments, the peelable tube 300 is used in disposable therapeutic solution delivery systems, such as those used outside of medical facilities. As an example, in-home peritoneal dialysis systems use a medical solution container 50, and tubing 310, among other components. The peelable tube 300 can be used to seal the medical solution inside the medical solution container 50, where the peelable tube 300 provides sufficient security from spillage during packaging and transit until the medical solution container 50 is delivered to the end user. The end user can prepare the system for use, and by simply pinching the heat seal 320, the medical solution flows into the system, and the treatment can begin. The peelable tube 300 is preferable to alternate forms of single use valves, such as frangible caps, which generate additional unwanted waste products, require greater amounts of coordination to operate, incur greater production costs, and other disadvantages.

In certain embodiments, the peelable tube 300 provides a sterile barrier between the contents of the medical solution container 50 and the outside environment. The heat seal 320 is configured to be airtight, watertight, and sealed sufficiently such that outside microbes and particulates cannot enter the medical solution container 50 via the peelable tube 300, and otherwise maintains a state of sterility prior to use.

According to some embodiments, the peelable tube 300 is a single use valve on a medical solution bag, such as a form seal fill (FSF) bag.

According to some embodiments, the fluid contained in the medical solution container 50 may be a saline solution, a therapeutic agent, blood, or serum.

The length X120 of the heat seal 320 is a functionally relevant dimension, as the strength of the heat seal 320 increases as the length X320 increases. As the length X320 of the heat seal 320 increases, so too increases the compressive force 350 required to compromise the seal 320 to open the peelable tube 300. Therefore, the length X320 of the heat seal 320 corresponds to both the gauge, and outer diameter D310, of the tube 310, and the volume of fluid the medical solution container 50, or other connected reservoir, is configured to contain. At the same time, the length X320 of the heat seal 320 may be configured such that the heat seal 320 does not require excessive force to disengage.

According to some embodiments, the ratio of the length X320 of the heat seal 320 to the outside diameter D310 of the tube 310 is about 3:1, about 2:1, about 3:2, about 1:1, about 2:3, about 1:2, or about 1:3.

Referring now to FIGS. 17-21, new and improved PD therapy system 40 preferably includes a new and improved distendable drain reservoir 400, in which an enclosed volume that may distend to accommodate a volume of drained liquid is provided. The distendable drain reservoir 400 is a user friendly, single use, stretchable reservoir that reduces material cost and environmental impact compared to traditional drain reservoir systems currently used with medical solution delivery systems. Production advantages over traditional drain reservoirs are provided by the distendable drain reservoir 400, such that production of the distendable drain reservoir 400 simply requires the production of a balloon-like vessel and a hard plastic ring. This is in contrast to traditional drain reservoirs, which require tubing, connectors, seals, and a relatively complex reservoir design to be manufactured, increasing complexity and materials consumed.

FIG. 17 shows a PD therapy system 40 with an empty distendable drain reservoir 400, according to embodiments of the present disclosure. The PD therapy system 40 has a supply of peritoneal dialysis solution in the ISBM container 50, a coil conduit 42, a patient connector 44, and a distendable drain reservoir 400 with a locking ring 402. Dialysis solution flows from the container 50, down coil connector 42 to a first input side of patient connector 44. Patient connector 44 interfaces with an PD cycler apparatus which supplies a biological subject with PD solution from the input side of patient connector 44 while allowing drain fluid from the biological subject to drain via the outlet side of patient connector 44. The distendable drain reservoir 400 is affixed to the drain conduit 404 with the locking ring 402, and the distendable drain reservoir 400 is kept in fluid communication with the outlet side of the patient connector 44.

The new and improved distendable drain reservoir 400, coil conduit 42, patient connector 44, drain conduit 404 and the locking ring 402 may be made of any appropriate material, including but not limited to plastic, latex, rubber, cellulose, composites, or combinations thereof preferably a polyolefin material. The distendable drain reservoir 400 may be made of any material sufficiently strong and elastic, and these characteristics may be chosen to permit the distendable drain reservoir 400 to stretch to accommodate an equivalent volume of PD solution which is initially present in the ISBM container 50. It will be appreciated that elements presented in the figures may be in different proportions than illustrated.

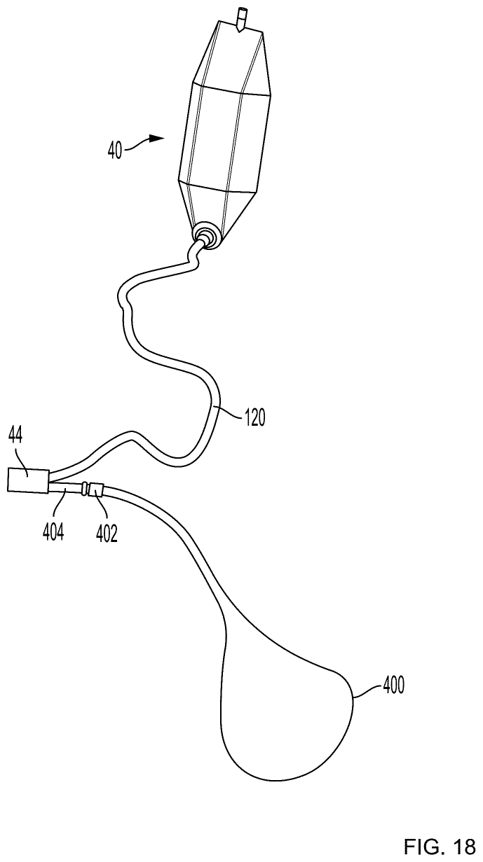

FIG. 18 shows a PD therapy system 40 with a distendable drain reservoir 400 containing drained fluid, according to embodiments of the present disclosure. Ingress of the drain fluid from the outlet side of the patient connector 44 causes the distendable drain reservoir 400 to distend. Distention of the distendable drain reservoir 400 is driven by a gravitational force acting upon the drain fluid, causing a heightened fluid pressure applied to an inner surface at a bottom end of the distendable drain reservoir 400 and forcing walls of the distendable drain reservoir 400 to expand and to bulge outwards as illustrated.

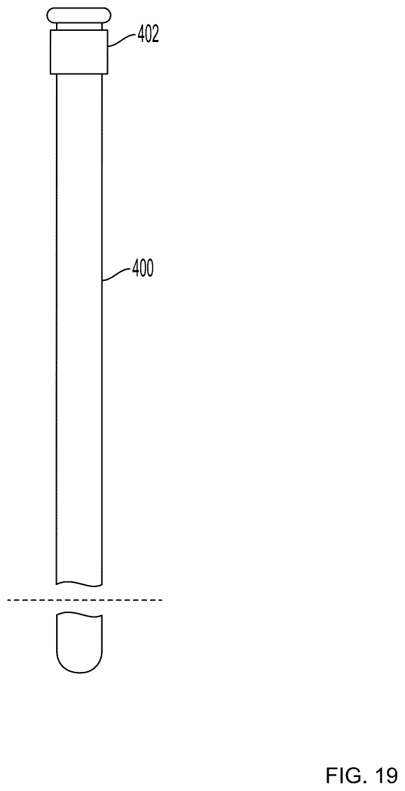

FIG. 19 shows a side profile view of a distendable drain reservoir 400 with a locking ring 402, according to embodiments of the present disclosure. The distendable drain reservoir 400 may have wall thicknesses selected for strength and elasticity. Walls which are too thick may not distend properly at fluid pressures achievable via gravity alone, while walls which are too thin may rupture under those same fluid pressures. For example, an example distendable drain reservoir 400 may be made from latex and may have wall thicknesses between 0.12 millimeters (mm) and 0.5 mm when empty. Filling the distendable drain reservoir 400 may cause the wall thicknesses to decrease drastically as the distendable drain reservoir 400 distends. For example, the example distendable drain reservoir 400 referenced above may, when full, have wall thicknesses between 0.012 mm and 0.05 mm. The example distendable drain reservoir 400 may have a length in the longitudinal dimension of between 25 mm and 400 mm.

The distendable drain reservoir 400 may comprise an envelope including a closed end portion, a port connection portion, and a distendable wall extending between the closed end portion and the port connection portion. The envelope may be watertight in a first configuration; having a first thickness of the distendable wall and defining a first volume between the port connection and the closed end portion described by the distendable wall. The envelope may also be watertight in a second configuration; having a second thickness of the distendable wall and defining a second volume between the port connection and the closed end portion described by the distendable wall. The first thickness may be greater than the second thickness while the first volume may be less than the second volume.

The distendable drain reservoir 400 may include a locking ring 402 configured to secure the port connection portion to a port or tube (e.g., the outlet side of patient connector 44 or drain conduit 46). The locking ring 402 may be a full ring, a C ring, a crimp ring, a spiral ring, or any other design of locking or retaining ring capable of securing a distendable drain reservoir to a port. The locking ring 402 may also be made of any rigid material capable of being fashioned into a ring shape, including but not limited to plastic, metal, cellulose, glass, silicone, or wood. For example, a locking ring 402 may be made from silicone and may have wall thicknesses between 0.5 mm and 10 mm and an inner diameter between 1 mm and 25 mm. The locking ring may have a length in the longitudinal dimension of between 4 mm and 50 mm.

The distendable drain reservoir 400 may have a shape that is either substantially tubular, wherein an undistended cross section cut from a transverse plane of the distendable drain reservoir 400 is substantially similar in diameter and shape along a majority (e.g., at least 90%) of the drain reservoir's longitudinal length and where the longitudinal length of the distendable drain reservoir 400 increases when the distendable drain reservoir 400 distends, or substantially globular, where the undistended cross section cut from the transverse plane widens then narrows in diameter as one moves away from an opening of the distendable drain reservoir 400. Globular embodiments may include a neck or stem of constant diameter adjacent to the opening. A lip may be provided at an opening of the port connection portion of the distendable drain reservoir 400, which may prevent the distendable drain reservoir 400 from slipping underneath the locking ring 402. The lip may comprise a bead of material disposed around an outer edge of the port connection portion. The lip may be fashioned from a same material as that of other portions of the distendable drain reservoir 400.

Unlike traditional drain bags, which have a fixed internal volume, the distendable drain reservoir 440 offers a variable volume. Although traditional drain bags may include accordion joints or other expansion features that fold/unfold to save of space when in an unfilled configuration, these drain bags must include sufficient material to define those expansion features in addition to the walls and support structures of the bag. These expansion features add additional weight and manufacturing complexity to the drain bags, and are often a point of failure due to faults in joints or weakness/fatigue at folded sections. Additionally, drain bags may often be over-dimensioned to avoid potential overflow during use, thereby leading to material waste. In contrast, the distendable drain reservoir 400 has a first volume in an unused (e.g., un-distended) state and a second volume in a used (e.g., distended) state, and the second volume adjusts to match the volume of the drain fluid in the distendable drain reservoir 400. The second volume changes by stretching the material from which the distendable drain reservoir 400 is made. The second volume of the distendable drain reservoir 400 (e.g., in the distended state) may correspond to a volume of drain fluid held in the distendable drain reservoir 400, which may in turn correspond to a volume of ISBM container 50.

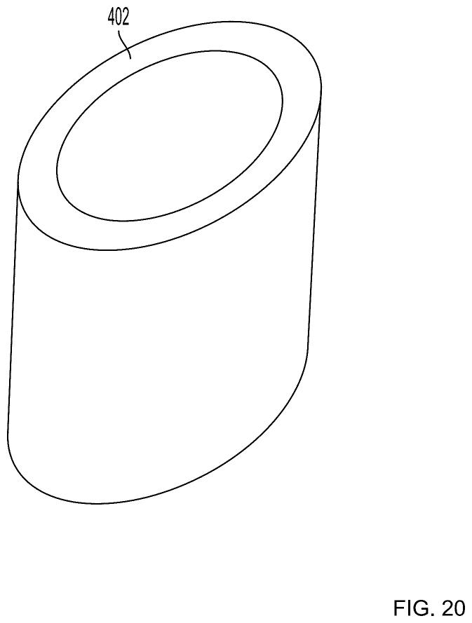

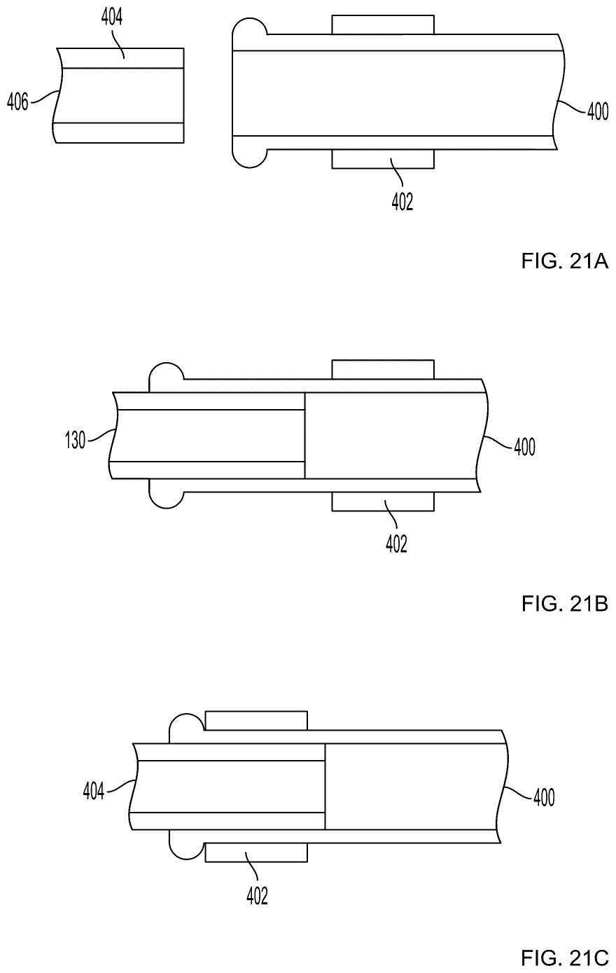

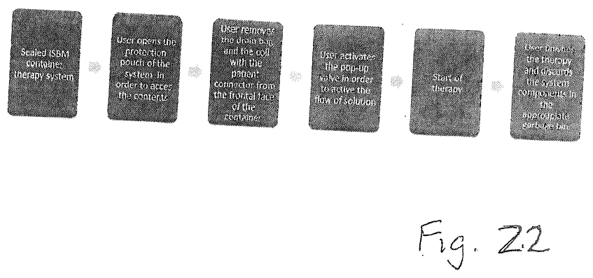

FIG. 20 illustrates an isometric view of a locking ring 402, according to embodiments of the present disclosure. The locking ring 402 may be a full ring, a C ring, a crimp ring, a spiral ring, or any other design of locking or retaining ring capable of securing a distendable drain reservoir (400) to a port (see FIG. 21A, FIG. 21B, and FIG. 21C). The locking ring 402 may also be made of any rigid material capable of being fashioned into a ring shape, including but not limited to plastic, metal, cellulose, glass, silicone, or wood. For example, a locking ring 402 may be made from acrylonitrile butadiene styrene and may have wall thicknesses between 0.5 mm and 10 mm and an inner diameter between 1 mm and 25 mm. The locking ring 402 may have a length in the longitudinal dimension of between 4 mm and 50 mm.

FIG. 21A illustrates a section view of a connector 406 at the end of the drain conduit 404, a distendable drain reservoir 400, and a locking ring 402 prior to installation (or after removal) of the distendable drain reservoir 400, according to embodiments of the present disclosure. The drain conduit 404 is adjacent to the distendable drain reservoir 400 with the locking ring 402 circumferentially disposed around an outer surface of the distendable drain reservoir 400.

FIG. 21B illustrates the section view of FIG. 21A during installation of a distendable drain reservoir 400, according to embodiments of the present disclosure. The distendable drain reservoir 400 is circumferentially disposed around an outer surface of the end of the drain conduit. A locking ring 402 is in turn circumferentially disposed around an outer surface of the distendable drain reservoir 400 but is positioned such that the locking ring 402 does not overlap the end of the drain conduit along a longitudinal axis of the distendable drain reservoir 400. The distendable drain reservoir 400 is thus not secured adequately to the drain conduit and may slide off of the drain conduit. The distendable drain reservoir 400 and the locking ring 402 may alternatively engage with a tube or any other connector structure capable of fitting within the distendable drain reservoir 400 and the locking ring 402.

FIG. 21C illustrates the section view of FIG. 21A following installation of a distendable drain reservoir 400, according to embodiments of the present disclosure. The distendable drain reservoir 400 is circumferentially disposed around an outer surface of the end of the drain conduit. A locking ring 402 is in turn circumferentially disposed around an outer surface of the distendable drain reservoir 400. The locking ring 402 is positioned to overlap the drain conduit end in order to secure the distendable drain reservoir 400 to the drain conduit, causing the drain conduit and the locking ring 402 to exert a radial clamping force on the distendable drain reservoir 400 and to form a watertight seal between an interior space of the drain conduit/distendable drain reservoir 400 and all spaces outside of the drain conduit/distendable drain reservoir 400. In some embodiments, the end of the drain conduit may have a raised lip or ridge circumferentially disposed around an outer edge of the drain conduit end, positioned at an end of drain conduit such that when the locking ring 402 is positioned along the distendable drain reservoir 400 to fully overlap the end of the drain conduit, the lip distends the distendable drain reservoir 400 outwards on an end of the locking ring 402 longitudinally opposite to an opening of the distendable drain reservoir 400 and engages with an end surface of the locking ring 402 to better secure the locking ring 402 in place.

FIG. 22 is a schematic flow chart depicting the steps of using the PD therapy systems 40 of this disclosure. The steps include providing a sealed ISBM container containing PD therapy solution, opening the pouch of the kit and connecting each of the kit parts. Thereafter the pop-up valve or outlet device is activated to commence flow of solution so that therapy may be started. At the conclusion of the therapy, the system components may be discarded in a garbage bin before being delivered to a recycling center.

Although this disclosure has been described in terms of preferred embodiments, modifications or changes will be suggested to those skilled in this art. The pop-up valve with the foil layer is preferred, but any of the other embodiments for the pop-up valve described in the related co-filed pop-up valve application may be substituted herein.

What is claimed is:

1. A fluid container for medical solutions comprising:
a hollow container body having a convex polyhedral configuration including a first half container body and a second opposing half container body disposed symmetrically about a central plane, each said half container body comprising a plurality of hingeably edge-connected body panels including four opposing angled side panels and four opposing angled corner panels, each side panel being hingeably edge-connected to a rectangular face panel, the hollow container body having an upper end and an opposed lower end, the upper end further including an injection site in the upper end; and one of the side panels adjacent the lower end of the container body further including an outlet valve-receiving shaped outlet orifice.

2. A fluid container for medical solutions as defined in claim 1, wherein the hollow container body is a unitary shaped molded article.

3. A fluid container for medical solutions as defined in claim 1, wherein the outlet valve-receiving shaped outlet orifice comprises a cylindrical portion defining a mounting surface for receiving an outlet pop-up valve.

4. A fluid container for medical solutions as defined in claim 1, wherein the container body is designed to be self-collapsing from the upper end to the lower end on emptying.

5. A fluid container for medical solutions as defined in claim 1, wherein the container body comprises a moldable polyolefin material.

6. A fluid container for medical solutions as defined in claim 1, wherein the container body comprises polypropylene.

7. A fluid container for medical solutions as defined in claim 1, wherein the container body comprises polyethylene.

8. A fluid container as defined in claim 1, wherein each side panel is rectangular and each corner panel is triangular.

9. A fluid container as defined in claim 1, further comprising a peel seal subdividing the interior of the container body to provide a first chamber and a second chamber which peel seal may be opened by application of force on at least one of the chambers to permit separately housed components to mix together prior to administration of fluid therapy.

10. A peritoneal dialysis therapy container comprising: a hollow container body having a convex polyhedral configuration including a first half container body and a second opposing half container body disposed symmetrically about a central plane, each said half container body comprising a plurality of hingeably edge-connected body panels including four opposing angled side panels and four opposing angled corner panels, each side panel being hingeably edge-connected to a rectangular face panel, the hollow container body having an upper end, an opposed lower end, and a fluid-receiving chamber, the upper end further including an injection site sealingly mounted into the upper end; and one of the side panels adjacent the lower end of the container body further including an outlet valve-receiving shaped outlet orifice; and
an outlet device comprising an outlet base member having an elongate cylindrical body having a peripheral radial mounting flange at one end and an opposed distal end with a peripheral radial retention barb and having an inlet passageway extending therethrough, the outlet base member being sealingly mounted in the outlet orifice; and
a peelable tube said peelable tube comprising an elongate tube having first and second opposed ends, an inner fluid passageway and an intermediate crimped heat seal section, the first end being sealingly mounted to the distal end of the outlet base member,
whereby application of a compressive force perpendicularly to the extremities of the heat seal section is effective to open the crimped heat seal so that fluid may flow from the fluid-receiving chamber through the inlet passageway and out through the second end.

11. A peritoneal dialysis therapy container as defined in claim 10, wherein the hollow container body is a unitary shaped molded article.

12. A peritoneal dialysis therapy container as defined in claim 10, wherein the outlet valve-receiving shaped outlet orifice comprises a cylindrical portion including a mounting surface for receiving the mounting flange of the outlet base member.

13. A peritoneal dialysis therapy container as defined in claim 10, wherein the container body is designed to be self-collapsing from the upper end to the lower end on emptying.

14. A peritoneal dialysis therapy container as defined in claim 10, wherein the container body comprises a moldable polyolefin material.

15. A peritoneal dialysis therapy container as defined in claim 10 wherein the container body comprises polypropylene.

16. A peritoneal dialysis therapy container as defined in claim 10, wherein the container body comprises polyethylene.

17. A peritoneal dialysis therapy container as defined in claim 10, further comprising a peel seal subdividing the fluid receiving chamber to provide a first chamber and a second chamber, which peel seal may be opened by application of force on at least one of the chambers to permit separately housed components to mix together prior to administration of fluid therapy.

18. A peritoneal dialysis therapy container as defined in claim 10, wherein the outer end of the shaft includes a luer connector for connecting the container and outlet valve to a coil conduit.

19. A peritoneal dialysis therapy container as defined in claim 10 wherein the pop-up valve is a one piece molded part.

20. A peritoneal dialysis therapy container as defined in claim 10 wherein the pop-up valve comprises a thermoplastic elastomer.

21. A peritoneal dialysis therapy container as defined in claim 10 wherein the pop-up valve comprises polypropylene and polyamide.

22. A peritoneal dialysis therapy container as defined in claim 10 wherein the pop-up valve comprises a blend of about 75% by weight polypropylene and 25% by weight polyamide.

23. A peritoneal dialysis therapy container as defined in claim 10 wherein the peelable tube includes a crimped heat-sealed section intermediate the length of the peelable tube which places the peelable tube in a closed configuration that blocks fluid communication between the first end and the second end, wherein the crimped heat seal is configured to disengage and return the tube to an open configuration when a compressive force is applied to the crimped heat sealed section in a direction perpendicular to a longitudinal axis of the peelable tube.

24. A peritoneal dialysis therapy container as defined in claim 23, wherein the crimped heat seal is defined such that an inner layer of the tube is compressed together, and forms a thermal bond around an inner diameter of the tube.

25. A peritoneal dialysis therapy container as defined in claim 23, wherein the crimped heat seal is configured to withstand at least about 100 kilopascals (kPa), at least 250 kPa, at least 500 kPa, or at least 1 megapascal (MPa) of fluid pressure within the tube.

26. A peritoneal dialysis therapy container as defined in claim 23, wherein a ratio of a magnitude of a minimum compressive force required to change the inlet port tube to the open configuration to an internal fluid pressure rating per square centimeter of the crimp heat seal is about 1:25, about 1:50, about 1:100, or about 1:250.

27. A peritoneal dialysis therapy container as defined in claim 23, wherein the inlet port tube contains an inner lining configured to bond with heat, such that the crimped heat seal is defined by a crimping action applied to the tube with heat.

28. A peritoneal dialysis therapy container as defined in claim 23, wherein the inlet port tube or inner lining contains a thermochromic element, such that when the crimped heat seal is formed, the crimped heat seal is indicated by a change in color state.

29. A peritoneal dialysis therapy container as defined in claim 23, wherein a ratio of a length of the crimped heat seal, along a longitudinal axis of the inlet tube, to a length of an outer diameter of the tube is about 2:1, about 3:2, about 1:1, about 2:3, or about 1:2.

30. A kit for a peritoneal dialysis therapy system comprising:
a solution container containing a peritoneal dialysis therapy solution; a pop-up outlet valve attached to the solution container; an injection site attached to the container;
a coil conduit having a first end attached to the pop-up outlet valve and an opposed end with a patient connector, the patient connector including a solution inlet conduit connected to the coil conduit and a drain conduit extending from the patient connector; and a drain bag having an internal drain reservoir connected to the drain conduit,
said solution container comprising: a hollow container body having a convex polyhedral configuration including a first half container body and a second opposing half container body disposed symmetrically about a central plane, each said half container body comprising a plurality of hingeably edge-connected body panels including four opposing angled rectangular side panels and four opposing angled triangular corner panels, each side panel being hingeably edge-connected to a rectangular face panel, the hollow container body having an upper end, an opposed lower end, and a fluid-receiving chamber, the upper end further including said injection site sealingly mounted into the upper end,
a pop-up outlet valve sealingly mounted in the outlet orifice, the pop-up outlet valve comprising a valve body extending axially between a container contacting end having a first diametrical dimension with an annular face and a central opening leading to an interior cavity and an opposed top wall extending parallel to and spaced from the annular face having a second smaller diametrical dimension, a double ended elongate cylindrical shaft with a central fluid passageway extending axially through the top wall from an inner end having an inner end face disposed in the cavity at a point intermediate the annular face and the top wall to an opposed outer end spaced from the top wall outside the cavity, the inner end including at least one window opening communicating with the central passageway, the valve body including a foldable collapsible peripheral sidewall bounding the cavity extending from the container contacting end to the top wall, the sidewall being movable between an inwardly folded closed position in which the inner end face is positioned adjacent the annular face in the central opening and an extended open position in which the end face is spaced inwardly away from the annular face, the pop-up valve further including a foil welded to the annular face and the inner end face in the closed position covering the central opening, the pop-up outlet valve being sealably welded to the outlet orifice, whereby the pop-up valve in the closed position closes off the outlet orifice and movement of the valve body from the closed position to the open position is effective to rupture the foil between the annular face and the inner end face, thereby permitting fluid to flow from the container through the outlet orifice, through the window opening into the central passageway to the outer end;
said drain bag being secured to the drain conduit via a locking ring, wherein a wall of the drain bag is configured to expand in surface area responsive to an outward fluid pressure upon an interior surface of the drain reservoir to allow the drain bag to distend to accommodate fluid from drain conduit.

31. A kit as defined in claim 30 further comprising a foil overpouch containing the solution container with outlet pop-up valve and the coil conduit with attached patient connector and the drain bag and locking ring.

32. A kit as defined in claim 31, wherein the foil overpouch and its contents are sterilized and are maintained in sterile condition until the foil overpouch is opened.

33. A kit as defined in claim 30 wherein each of the parts comprises a polyolefin material.

34. A kit as defined in claim 30 wherein each of the parts comprises a polypropylene material.

35. A kit for a peritoneal dialysis therapy system comprising:
a solution container containing a peritoneal dialysis therapy solution; an outlet delivery device and an injection site attached to the solution container;
a coil conduit having a first end attached to the outlet delivery device and an opposed end with a patient connector, the patient connector including a solution inlet conduit connected to the coil conduit and a drain conduit extending from the patient connector; and a drain bag having an internal drain reservoir connected to the drain conduit,
said solution container comprising: a hollow container body having a convex polyhedral configuration including a first half container body and a second opposing half container body disposed symmetrically about a central plane, each said half container body comprising a plurality of hingeably edge-connected body panels including four opposing angled rectangular side panels and four opposing angled triangular corner panels, each side panel being hingeably edge-connected to a rectangular face panel, the hollow container body having an upper end, an opposed lower end, and a fluid-receiving chamber, the upper end further including said injection site mounted in the upper end; the outlet delivery device comprising an outlet base insert having an elongate cylindrical body having a peripheral radial mounting flange at one end and an opposed distal end with a peripheral radial retention barb and having an inlet passageway extending therethrough, said outlet base insert being sealingly mounted in the outlet orifice by welding the mounting flange to a mounting surface defined by the outlet orifice to seal the outlet base insert to the solution container;
said outlet delivery device further comprising a peelable tube comprising an elongate tube having first and second opposed ends, an inner fluid passageway and a crimped heat seal section disposed between the first and second ends, the first end of the peelable tube being telescopically received over the barbed distal end of the outlet base insert, and the crimped heat seal section and the opposed second end project outwardly from the lower end,
said drain bag being secured to the drain conduit via a locking ring, wherein a wall of the drain bag is configured to expand in surface area responsive to an outward fluid pressure upon an interior surface of the drain reservoir to allow the drain bag to distend to accommodate drain fluid from the patient connector and drain conduit,
whereby, the heat seal section can be uncrimped and opened to permit container solution to flow out of the orifice and into the outlet base insert and peelable tube which may in turn be connected to a coil conduit.

36. A kit as defined in claim 35 further comprising a foil overpouch containing the solution container with outlet pop-up valve, and the coil conduit with attached patient connector and the drain bag and locking ring.

37. A kit as defined in claim 36, wherein the foil overpouch and its contents are sterilized and are maintained in sterile condition until the foil overpouch is opened.

38. A kit as defined in claim 35 wherein each of the parts comprises a polyolefin material.

39. A kit as defined in claim 35 wherein each of the parts comprises a polypropylene material.
